(19) United States
(12) Patent Application Publication
(10) Pub. No.: US 2012/0283485 A1
(43) Pub. Date: Nov. 8, 2012

(54) ROBUST PROMOTER CATALYST SYSTEM

(76) Inventors: Umesh Krishna Hasyagar, Bangalore (IN); Rathinam Jothi Mahalingam, Bangalore (IN); Kishan Gurram, Bangalore (IN); Paul Eijsbouts, Nieuwkuijk (NL)

(21) Appl. No.: 13/099,032

(22) Filed: May 2, 2011

(51) Int. Cl. C07C 39/16; B01J 41/12; B01J 31/10

(52) U.S. Cl. 568/728; 502/159; 521/30

(57) ABSTRACT

A modified ion exchange resin catalyst having an attached dimethyl thiazolidine promoter is disclosed. Also disclosed is a process for catalyzing condensation reactions between phenols and ketones, wherein reactants are contacted with a modified ion exchange resin catalyst having an attached dimethyl thiazolidine promoter. Also disclosed is a process for catalyzing condensation reactions between phenols and ketones that does not utilize a bulk promoter.

### BACKGROUND

1. Technical Field

The present disclosure relates to catalyst systems, and specifically to promoter ion exchange resin catalyst systems.

2. Technical Background

Many conventional condensation reactions utilize inorganic acid catalysts, such as sulfuric acid or hydrochloric acid. Use of such inorganic acid catalysts can result in the formation of undesirable byproducts that must be separated from the reaction stream. Ion exchange resin catalyst systems can also be used, but the inherent low acid concentration can require the use of a promoter or rate accelerator.

When used as part of the catalyst system, reaction promoters can improve reaction rate and selectivity. In the case of the condensation of phenol and ketone to form bisphenol-A (BPA), reaction promoters can improve selectivity for the desired para-para BPA isomer.

Reaction promoters can be used as bulk promoters, where the promoter is present as an unattached molecule in the reaction medium, or as an attached promoter, where the promoter is attached to portion of the catalyst system.

In the synthesis of BPA, the use of 3-mercaptopropionic acid (3-MPA) as a promoter can produce a significant quantity of the less desirable o,p-BPA isomer, as opposed to the preferred p,p-BPA isomer.

Existing attached promoter systems, such as, pyridyl ethylmercaptons (PEM), can be susceptible to reactant impurities. For example, in the production of BPA, hydroxyacetone (HA) and methanol can be present in phenol and acetone reactants, respectively. As impurities, such as HA and methanol, can deactivate promoter systems, additional process steps to remove the impurities can be required. Such attached promoter systems can also be susceptible to impurities in recycle feeds of reaction processes, reducing the lifetime and performance of the catalyst system.

While much effort has been applied to the development and use of bulk and attached promoter systems, a need still exists for a manufacturing process and promoter catalyst system that can provide improved reaction rates, improved selectivity, and exhibit an improved tolerance for impurities over conventional systems. Thus, there is a need to address these and other shortcomings associated with existing promoter catalyst systems. These needs and other needs are satisfied by the compositions and methods of the present disclosure.

### SUMMARY

In accordance with the purpose(s) of the invention, as embodied and broadly described herein, this disclosure, in one aspect, relates tocatalyst systems, and specifically to promoter ion exchange resin catalyst systems.

In one aspect, the present disclosure provides a catalyst system comprising a cross-linked, sulfonated ion exchange resin catalyst and a dimethyl thiazolidine promoter.

In another aspect, the present disclosure provides a catalyst system comprising a cross-linked, sulfonated ion exchange resin catalyst and a dimethyl thiazolidine promoter, wherein the cross-linked,sulfonated ion exchange resin comprises a plurality of sulfonic acid groups and has a degree of cross-linking of from about 1% to about 4%.

In another aspect, the present disclosure provides a catalyst system comprising a cross-linked, sulfonated ion exchange resin catalyst and a dimethyl thiazolidine promoter, wherein the dimethyl thiazolidine promoter is at least partially bound to the cross-linked, sulfonated ion exchange resin.

In another aspect, the present disclosure provides a catalyst system comprising a cross-linked, sulfonated ion exchange resin catalyst and a dimethyl thiazolidine promoter, wherein the dimethyl thiazolidine promoter is bound to from about 18% to about 25% of the sulfonic acid groups of the cross-linked, sulfonated ion exchange resin.

In another aspect, the present disclosure provides an attached promoter catalyst system comprising an ion exchange resin and a dimethyl thiazolidine promoter, wherein the catalyst system is more resistant to hydroxyacetone than a conventional bulk promoter system.

In another aspect, the present disclosure provides a method for catalyzing a condensation reaction, the method comprising contacting two or more reactants with a modified ion exchange resin catalyst in the absence of a bulk promoter.

In another aspect, the present disclosure provides a method for catalyzing a condensation reaction, the method comprising contacting two or more reactants with a modified ion exchange resin catalyst in the absence of a bulk promoter, wherein the modified ion exchange resin catalyst comprises a cross-linked, sulfonated ion exchange resin.

In another aspect, the present disclosure provides a method for catalyzing a condensation reaction, the method comprising contacting two or more reactants with a modified ion exchange resin catalyst in the absence of a bulk promoter, wherein the modified ion exchange resin catalyst comprises an attached dimethyl thiazolidine promoter.

In another aspect, the present disclosure provides a method for the production of bisphenol-A, the method comprising contact a phenol and at least one of a ketone, an aldehyde, or a combination thereof in the presence of an attached ion exchange resin catalyst comprising a dimethyl thiazolidine promoter, wherein the method does not comprise a pretreatment and/or purification step for the phenol, ketone, and/or aldehyde.

### BRIEF DESCRIPTION OF THE FIGURES

The accompanying figures, which are incorporated in and constitute a part of this specification, illustrate several aspects and together with the description serve to explain the principles of the invention.

FIG. 1 illustrates a comparison of p,p-BPA formation using an inventive catalyst, both with and without hydroxyacetone present.

FIG. 2 represents data from a methanol spiking experiment with the inventive catalyst system, illustrating the formation of p,p-BPA over time in the presence of methanol.

FIG. 3 represents data from a methanol spiking experiment with the inventive catalyst system, illustrating catalyst selectivity over time in the presence of methanol.

FIG. 4 represents data from a methanol spiking experiment with the inventive catalyst system, illustrating catalyst selectivity vs. methanol concentration.

FIG. 5 represents data from a methanol spiking experiment with the inventive catalyst system, illustrating p,p-BPA formation in the presence of varying methanol concentration.

Additional aspects of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such can, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, example methods and materials are now described.

As used in the specification and the appended claims, the singular forms “a,” “an” and “the” include plural referents unless the context clearly dictates otherwise. Thus, for example, reference to “a ketone” includes mixtures of two or more ketones.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “optional” or “optionally” means that the subsequently described event or circumstance can or can not occur, and that the description includes instances where said event or circumstance occurs and instances where it does not. For example, the phrase “optionally substituted alkyl” means that the alkyl group can or can not be substituted and that the description includes both substituted and unsubstituted alkyl groups.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds can not be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

References in the specification and concluding claims to parts by weight of a particular element or component in a composition or article denote the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

The term “alkyl group” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is an alkyl group containing from one to six carbon atoms.

The term “alkoxy” as used herein is an alkyl group bound through a single, terminal ether linkage; that is, an “alkoxy” group may be defined as —OR where R is alkyl as defined above. A “lower alkoxy” group is an alkoxy group containing from one to six carbon atoms.

The term “alkenyl group” as used herein is a hydrocarbon group of from 2 to 24 carbon atoms and structural formula containing at least one carbon-carbon double bond. Asymmetric structures such as (AB)C═C(CD) are intended to include both the E and Z isomers. This may be presumed in structural formulae herein wherein an asymmetric alkene is present, or it may be explicitly indicated by the bond symbol C.

The term “alkynyl group” as used herein is a hydrocarbon group of 2 to 24 carbon atoms and a structural formula containing at least one carbon-carbon triple bond.

The term “aryl group” as used herein is any carbon-based aromatic group including, but not limited to, benzene, naphthalene, etc. The term “aromatic” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorus. The aryl group can be substituted or unsubstituted. The aryl group can be substituted with one or more groups including, but not limited to, alkyl, alkynyl, alkenyl, aryl, halide, nitro, amino, ester, ketone, aldehyde, hydroxy, carboxylic acid, or alkoxy.

The term “cycloalkyl group” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl group” is a cycloalkyl group as defined above where at least one of the carbon atoms of the ring is substituted with a heteroatom such as, but not limited to, nitrogen, oxygen, sulphur, or phosphorus.

The term “aralkyl” as used herein is an aryl group having an alkyl, alkynyl, or alkenyl group as defined above attached to the aromatic group. An example of an aralkyl group is a benzyl group.

The term “hydroxyalkyl group” as used herein is an alkyl, alkenyl, alkynyl, aryl, aralkyl, cycloalkyl, halogenated alkyl, or heterocycloalkyl group described above that has at least one hydrogen atom substituted with a hydroxyl group.

The term “alkoxyalkyl group” is defined as an alkyl, alkenyl, alkynyl, aryl, aralkyl, cycloalkyl, halogenated alkyl, or heterocycloalkyl group described above that has at least one hydrogen atom substituted with an alkoxy group described above.

The term “ester” as used herein is represented by the formula —C(O)OA, where A can be an alkyl, halogenated alkyl, alkenyl, alkynyl, aryl, heteroaryl, cycloalkyl, cycloalkenyl, heterocycloalkyl, or heterocycloalkenyl group described above.

The term “carbonate group” as used herein is represented by the formula —OC(O)OR, where R can be hydrogen, an alkyl, alkenyl, alkynyl, aryl, aralkyl, cycloalkyl, halogenated alkyl, or heterocycloalkyl group described above.

The term “carboxylic acid” as used herein is represented by the formula —C(O)OH.

The term “aldehyde” as used herein is represented by the formula —C(O)H.

The term “keto group” as used herein is represented by the formula —C(O)R, where R is an alkyl, alkenyl, alkynyl, aryl, aralkyl, cycloalkyl, halogenated alkyl, or heterocycloalkyl group described above.

The term “carbonyl group” as used herein is represented by the formula C═O.

The term “ether” as used herein is represented by the formula AOA¹, where A and A¹ can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “sulfo-oxo group” as used herein is represented by the formulas —S(O)₂R, —OS(O)₂R, or, —OS(O)₂OR, where R can be hydrogen, an alkyl, alkenyl, alkynyl, aryl, aralkyl, cycloalkyl, halogenated alkyl, or heterocycloalkyl group described above.

Each of the materials disclosed herein are either commercially available and/or the methods for the production thereof are known to those of skill in the art.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

As briefly described above, the present disclosure provides a manufacturing process and a promoter catalyst system that can be useful in condensation reactions, such as, for example, the synthesis of bisphenol-A (BPA). BPA can be synthesized by the acid catalyzed condensation of phenol and acetone using either an HCl catalyst or a sulphonated ion exchange resin (IER) catalyst. Due to the inherent low number of acid sites on conventional ion exchange resins, IER processes typically incorporate a promoter system to improve reaction rates. Promoter systems can be bulk, wherein the promoter species is disposed in the reaction medium, or attached, wherein the promoter species is attached to another portion of the catalyst system.

A conventional IER based process utilizes 3-mercaptopropionic acid (3-MPA) as a bulk promoter. While bulk promoters can improve the reaction rate, they require recovery of the promoter species and typically do not provide a high degree of selectivity. For example, in the production of BPA, the use of a 3-MPA promoter can provide a wide range of BPA isomers. Specifically, 3-MPA based systems can result in the production of a significant quantity of o,p-BPA, as opposed to more desirable p,p-BPA. As such, separate isomerization reactions can be necessary to convert o,p-BPA to the more desirable p,p-BPA.

Alternatively, promoter systems can be attached, wherein the promoter is attached to portion of the catalyst system, such as the ion exchange resin. An exemplary attached promoter system utilizes a pyridyl ethylmercapton (PEM) promoter. Conventional attached promoter catalyst systems, such as a PEM based system, can be sensitive to impurities in reactant and recycle streams. For example, in the production of BPA, phenol and acetone reactants can contain impurities such as hydroxyacetone (HA) and methanol, respectively. These impurities can deactivate the catalyst system, resulting in slower reaction rates and shorter catalyst lifetimes.

In one aspect, the present disclosure provides a promoter catalyst system that is more selective than conventional promoter catalyst systems. In another aspect, the present disclosure provides a manufacturing process and catalyst system for the production of BPA that can selectively produce p,p-BPA without necessitating additional isomerizations reactions. In another aspect, the present disclosure provides a promoter catalyst system that can tolerate impurities, such as hydroxyacetone and methanol, in reactant and/or recycle streams.

In one aspect, the methods described here can be useful for the preparation of BPA. It should also be noted that reactants for bisphenol condensation reactions can comprise phenols, ketones and/or aldehydes, or mixtures thereof. In one aspect, any specific recitation of a ketone, such as acetone, or an aldehyde, is intended to include aspects where only the recited species is used, aspects wherein the other species (e.g., aldehyde for ketone) is used, and aspects wherein a combination of species is used. In other aspects, the methods described herein can be useful for the preparation of other chemical species from, for example, condensation reactions.

In one aspect, phenol reactants can comprise an aromatic hydroxy compound having at least one unsubstituted position, and optionally one or more inert substituents such as hydrocarbyl or halogen at one or more ring positions. In one aspect, an inert substituent is a substituent which does not interfere undesirably with the condensation of the phenol and ketone or aldehyde and which is not, itself, catalytic. In another aspect, phenol reactants are unsubstituted in the position para to the hydroxyl group. As recited here, hydrocarbyl functionalities comprise carbon and hydrogen atoms, such as, for example, alkylene, alkyl, cycloaliphatic, aryl, arylene, alkylarylene, arylalkylene, alkylcycloaliphatic and alkylenecycloaliphatic are hydrocarbyl functions, that is, functions containing carbon and hydrogen atoms.

In one aspect, an alkyl group, if present in a phenol species, comprises from 1 to about 20 carbon atoms, or from 1 to about 5 carbon atoms, or from 1 to about 3 carbon atoms, such as, for example, various methyl, ethyl, propyl, butyl and pentyl isomers. In one aspect, alkyl, aryl, alkaryl and aralkyl substituents are suitable hydrocarbyl substituents on the phenol reactant.

In one aspect, other inert phenol substituents can include, but are not limited to alkoxy, aryloxy or alkaryloxy, wherein alkoxy includes methoxy, ethoxy, propyloxy, butoxy, pentoxy, hexoxy, heptoxy, octyloxy, nonyloxy, decyloxy and polyoxyethylene, as well as higher homologues; aryloxy, phenoxy, biphenoxy, naphthyloxy, etc. and alkaryloxy includes alkyl, alkenyl and alkylnyl-substituted phenolics. Additional inert phenol substituents can include halo, such as bromo, chloro or iodo.

While not intending to be limiting, exemplary phenols can comprise, phenol, 2-cresol, 3-cresol, 4-cresol, 2-chlorophenol, 3-chlorophenol, 4-chlorophenol, 2-tert-butylphenol, 2,4-dimethylphenol, 2-ethyl-6-methylphenol, 2-bromophenol, 2-fluorophenol, 2-phenoxyphenol, 3-methoxyphenol, 2,3,6-trimethylphenol, 2,3,5,6-tetramethylphenol, 2,6-xylenol, 2,6-dichlorophenol, 3,5-diethylphenol, 2-benzylphenol, 2,6-di-tertbutylphenol, 2-phenylphenol, 1-naphthol, 2-naphthol, and/or combinations thereof. In another aspect, phenol reactants can comprise phenol, 2- or 3-cresol, 2,6-dimethylphenol, resorcinol, naphthols, and/or combinations or mixtures thereof. In one aspect, a phenol is unsubstituted.

In one aspect, the phenol starting materials can be commercial grade or better. As readily understood by one of ordinary skill in the art commercial grade reagents may contain measurable levels of typical impurities such as acetone, alpha-methylstyrene, acetophenone, alkyl benzenes, cumene, cresols, water, hydroxyacetone, methyl benzofuran, methyl cyclopentenone, and mesityl oxide, among others.

In one aspect, ketones, if used, can comprise any ketone having a single carbonyl (C═O) group or several carbonyl groups, and which are reactive under the conditions used. In another aspect, ketones can be substituted with substituents that are inert under the conditions used, such as, for example those inert substituents recited above with respect to phenols.

In one aspect, a ketone can comprise aliphatic, aromatic, alicyclic or mixed aromatic-aliphatic ketones, diketones or polyketones, of which acetone, methyl ethyl ketone, diethyl ketone, benzyl, acetyl acetone, methyl isopropyl ketone, methyl isobutyl ketone, acetophenone, ethyl phenyl ketone, cyclohexanone, cyclopentanone, benzophenone, fluorenone, indanone, 3,3,5-trimethylcyclohexanone, anthraquinone, 4-hydroxyacetophenone, acenaphthenequinone, quinone, benzoylacetone and diacetyl are representative examples. In another aspect, a ketone having halo, nitrile or nitro substituents can also be used, for example, 1,3-dichloroacetone or hexafluoroacetone.

Exemplary aliphatic ketones can comprise acetone, ethyl methyl ketone, isobutyl methyl ketone, 1,3-dichloroacetone, hexafluoroacetone, or combinations thereof. In one aspect, the ketone is acetone, which can condense with phenol to produce 2,2-bis-(4-hydroxyphenyl)-propane, commonly known as bisphenol A. In another aspect, a ketone comprises hexafluoroacetone, which can react with two moles of phenol to produce 2,2-bis-(4-hydroxyphenyl)-hexafluoropropane (bisphenol AF). In another aspect, a ketone can comprise a ketone having at least one hydrocarbyl group containing an aryl group, for example, a phenyl, tolyl, naphthyl, xylyl or 4-hydroxyphenyl group.

Other exemplary ketones can include 9-fluorenone, cyclohexanone, 3,3,5-trimethylcyclohexanone, indanone, indenone, anthraquinone, or combinations thereof. Still other exemplary ketones can include benzophenone, acetophenone, 4-hydroxyacetophenone, 4,4′-dihydroxybenzophenone, or combinations thereof.

In one aspect, a ketone reactant can be commercial grade or better. As readily understood by one of ordinary skill in the art commercial grade reagents may contain measurable levels of typical impurities such as aldehydes, acetophenone, benzene, cumene, diacetone alcohol, water, mesityl oxide, and methanol, among others. In one aspect, a ketone, such as, for example, acetone, has less than about 250 ppm of methanol. In another aspect, the inventive catalyst systems of the present invention can tolerate higher concentrations of impurities, such that a ketone can comprise more than 250 ppm of methanol.

In other aspects, the various methods and catalyst systems described herein can be used for the condensation of phenols with aldehydes, for example, with formaldehyde, acetaldehyde, propionaidehyde, butyraldehyde or higher homologues of the formula RCHO, wherein R is alkyl of, for example, 1 to 20 carbon atoms. In one aspect, the condensation of two moles of phenol with one mole of formaldehyde produces bis-(4-hydroxyphenyl)methane, also known as Bisphenol F. It should also be understood that dialdehydes and ketoaldehdyes, for example, glyoxal, phenylglyoxal or pyruvic aldehyde, can optionally be used.

### Promoter Catalyst System—Ion Exchange Resin

The promoter catalyst system of the present disclosure comprises an ion exchange resin catalyst and a promoter. In one aspect, the ion exchange resin can comprise any ion exchange resin suitable for use in the catalyst system of the present invention. In another aspect, the ion exchange resin comprises a cross-linked cationic exchange resin. In another aspect, the ion exchange resin comprises a cross-linked sulfonated ion exchange resin having a plurality of sulfonic acid sites. In yet another aspect, the ion exchange resin is acidic or strongly acidic. In one aspect, at least a portion of the ion exchange resin comprises sodium polystyrene sulfonate. In still other aspects, the ion exchange resin can comprise a monodispersed resin, a polydispersed resin, or a combination thereof.

The specific chemistry of an ion exchange resin or any one or more polymer materials that form a part of an ion exchange resin can vary, and one of skill in the art, in possession of this disclosure, could readily select an appropriate ion exchange resin. In one aspect, the ion exchange resin comprises polystyrene or a derivatized polystyrene. In another aspect, the ion exchange resin comprises a polysiloxane or derivatized polysiloxane. It should also be understood that the catalyst system can, in one aspect, comprise multiple ion exchange resins of the same or varying composition, acidity, and/or degree of cross-linking.

In one aspect, the ion exchange resin can be cross-linked with the same or a different polymer material. In various aspects, the degree of cross-linking is from about 1 percent to about 4 percent, for example, about 1, 1.2, 1.4, 1.6, 1.8, 2, 2.2, 2.4, 2.6, 2.8, 3, 3.2, 3.4, 3.6, 3.8, or 4 percent; or from about 1.5 percent to about 2.5 percent, for example, about 1.5, 1.6, 1.7, 1.8, 1.9, 2, 2.1, 2.2, 2.3, 2.4, or 2.5 percent. In other aspects, the degree of cross-linking can be less than 1 percent or greater than 4 percent, and the present invention is not intended to be limited to any particular degree of cross-linking recited here. In a specific aspect, the degree of cross-linking is about 2 percent. In another aspect, the ion exchange resin is not cross-linked. While not wishing to be bound by theory, cross-linking of an ion exchange resin is not necessary, but can provide additional stability to the resin and the resulting catalyst system.

In one aspect, the ion exchange resin can be cross-linked using any conventional cross-linking agents, such as, for example, polycyclic aromatic divinyl monomers, divinyl benzene, divinyl toluene, divinyl biphenyl monomers, or combinations thereof.

In other aspects, the ion exchange resin comprises a plurality of acid sites, and has, before modification, at least about 3, at least about 3.5, at least about 4, at least about 5, or more acid milliequivalents per gram (meq/g) when dry. In a specific aspect, the ion exchange resin, before modification, has at least about 3.5 acid milliquivalents per gram when dry. In various aspects, any of the plurality of acid sites on an ion exchange resin can comprise a sulfonic acid functionality, which upon deprotonation produces a sulfonate anion functionality, a phosphonic acid functionality, which upon deprotonation produces a phosphonate anion functionality, or a carboxylic acid functionality, which upon deprotonation produces a carboxylate anion functionality.

Exemplary ion exchange resins can include, but are not limited to, DIAION® SK104, DIAION® SK1B, DIAION® PK208, DIAION® PK212 and DIAION® PK216 (manufactured by Mitsubishi Chemical Industries, Limited), A-121, A-232, and A-131, (manufactured by Rohm & Haas), T-38, T-66 and T-3825 (manufactured by Thermax), LEWATIT® K1131, LEWATIT® K1221 (manufactured by Lanxess), DOWEX® 50W2X, DOWEX® 50W4X, DOWEX® 50W8X resins (manufactured by Dow Chemical), Indion 180, Indion 225 (manufactured by Ion Exchange India Limited), and PUROLITE® CT-222 and PUROLITE® CT-122 (manufactured by Purolite).

### Promoter Catalyst System—Promoter

In one aspect, the promoter of the present invention comprises dimethyl thiazolidine (DMT). In other aspects, the promoter of the present invention can comprise derivatives and/or analogues of dimethyl thiazolidine. In another aspect, the promoter of the present invention can be represented by the formula:

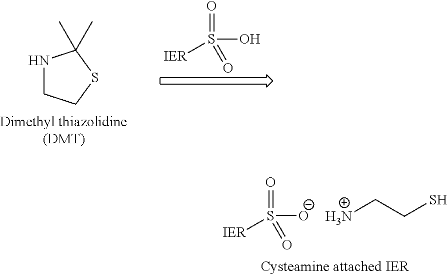

In one aspect, the promoter can be contacted with the ion exchange resin so as to neutralize at least a portion of the available acid sites on the ion exchange resin, and attach thereto. In various aspects, the ion exchange resin is modified by neutralizing from about 18% to about 25% of the available acid sites with the promoter. In another aspect, the promoter is bound to from about 18% to about 25%, for example, about 18, 19, 20, 21, 22, 23, 24, or 25% of the acid sites on the ion exchange resin. In another aspect, the promoter is bound to from about 20% to about 24% of the acid sites on the ion exchange resin. In still another aspect, the promoter is bound to about 22% of the acid sites of the ion exchange resin.

In an exemplary process, the promoter is combined with a solvent to form a mixture. The mixture may further comprise an acid to improve solubility of the promoter. In one aspect, the amount of acid can be sufficient to solubilize the promoter but not enough to impede modification of the ion exchange resin. In one aspect, the amount of acid is typically less than or equal to about 1 equivalent; or less than or equal to about 0.25 equivalents, based on the number of moles of the promoter. Exemplary acids include, but are not limited to, hydrochloric acid (HCl), p-toluenesulfonic acid, trifluorocacetic acid, and acetic acid. In such an aspect, the mixture can be contacted with the ion exchange resin resulting in an ionic linkage between the promoter cation and anion (deprotonated acid site) of the ion exchange resin. Formation of the ionic linkage can thus neutralize the acid site.

The degree of neutralization may be determined in a number of ways. In one aspect, the modified ion exchange resin catalyst can be titrated to determine the amount of remaining acid sites.

Following modification (neutralization), the modified ion exchange resin catalyst can optionally be rinsed with a continuous flow of phenol to remove any remaining amounts of solvent from the modification. Alternatively, if acid was used to improve the solubility of the promoter, the modified ion exchange resin can optionally be rinsed with deionized water prior to rinsing with phenol. In one aspect, removing substantially all of the water is herein defined as removing greater than or equal to about 75%, greater than or equal to about 80%, or greater than or equal to about 85%, based on the total amount of water initially employed.

In one aspect, at least a portion of the promoter is ionically bound to the available acid sites of the ion exchange resin. In another aspect, all or substantially all of the promoter is ionically bound to acid sites of the ion exchange resin. In another aspect, at least a portion of the promoter is covalently bound to at least a portion of the ion exchange resin. In still another aspect, all or substantially all of the promoter is at least covalently bound to the ion exchange resin. In yet another aspect, the degree of attachment or binding between a promoter and an ion exchange resin can vary, such as, for example, covalent binding, ionic binding, and/or other interactions or attraction forces, and the present invention is not intended to be limited to any particular degree of attachment.

### Reactant Impurities

For the manufacture of BPA, both phenol and acetone reactants can contain impurities, such as hydroxyacetone (HA) and methanol, respectively. These reactants can interfere with and/or deactivate catalyst systems, resulting in shortened catalyst lifetimes and/or decreased reaction rates. A conventional approach to prevent such deactivation is to subject the reactants to a pretreatment step, such as an adsorption bed, to remove the impurities.

In one aspect, the DMT attached promoter catalyst system of the present invention can tolerate phenol and alcohol impurities without reducing the lifetime of the catalyst system. In another aspect, the DMT attached promoter catalyst system can tolerate other impurities detrimental to conventional catalyst systems. In yet another aspect, the DMT attached promoter catalyst system can provide performance equivalent to or greater than that of conventional bulk promoter systems. In comparison with a conventional PEM attached promoter catalyst system, the DMT catalyst system can exhibit no significant change in catalyst activity level after exposure to HA. Thus, in one aspect, the DMT catalyst system can eliminate the need for separate purification and/or pretreatment steps.

In one aspect, a manufacturing process using the DMT catalyst system can require a reduced level of pretreatment and/or purification of reactants. In another aspect, a bisphenol manufacturing process can utilize phenol and acetone reactants as received, without the need for a pretreatment step. In still other aspects, the lifetime of a DMT promoter catalyst system, after exposed to HA and/or methanol, can be longer than that for conventional bulk or attached promoter catalyst systems.

In one aspect, the DMT catalyst system can tolerate a greater amount of hydroxyacetone than a comparative PEM catalyst system. In various aspects, upon exposure to about 10 ppm hydroxyacetone, the DMT catalyst system can maintain at least about 60, at least about 65, at least about 70, at least about 75, or at least about 80% of its initial performance after 200 hours of operation, in terms of the amount of p,p-BPA produced. In other aspects, upon exposure to about 10 ppm hydroxyacetone, the DMT catalyst system can maintain at least about 10, at least about 15, at least about 20, or at least about 25% of its initial performance after 500 hours of operation, in terms of the amount of p,p-BPA produced.

As described above, the DMT catalyst system can be more resistant to deactivation than other catalyst systems. In one aspect, the DMT catalyst system can substantially maintain its acid strength after 100 hours of operation under 20 ppm of hydroxyacetone. In various aspects, the acid strength (meq/g) of the DMT catalyst system, after 100 hours of exposure to 20 ppm hydroxyacetone, is within 10%, within 8%, within 6%, within 4%, or within 2% of the acid strength for a DMT catalyst system not exposed to hydroxyacetone. In a specific aspect, the acid strength of the DMT catalyst system, after 100 hours of exposure to 20 ppm hydroxyacetone, is within 5% of the acid strength for a DMT catalyst system not exposed to hydroxyacetone.

In addition to improved resistance to hydroxyacetone, the DMT catalyst system can tolerate exposure to alcohols, such as methanol, with substantially no change in performance. In various aspects, the DMT catalyst system can tolerate up to about 100 ppm, up to about 250 ppm, up to about 500 ppm, up to about 1,000 ppm, up to about 1,500 ppm, up to about 2,000 ppm, up to about 2,500 ppm, up to about 3,000 ppm, up to about 4,000 ppm, up to about 5,000 ppm, up to about 6,000, or more of methanol with no or substantially no detectable decrease in performance. In a specific aspect, the DMT catalyst system can maintain a production rate of p,p-BPA upon exposure to up to about 3,000 ppm methanol. In other aspects, exposure to methanol at each of the concentrations recited above, does not result in any significant change in the selectivity of the DMT catalyst system.

### Recycle Stream Impurities

In addition to reactant impurities, conventional attached promoter systems, such as pyridyl ethylmercaptons (PEM) are also susceptible to impurities in process recycle feeds. In conventional BPA manufacturing processes, a stream of about 10-12% BPA product is recycled to the main reactor, and can be combined with a quantity of fresh acetone. As with reactant impurities, conventional processes can utilize separate purification systems, such as adsorption beds, to remove recycle stream impurities and thus, prevent catalyst deactivation and improve catalyst lifetime.

In one aspect, the DMT attached promoter catalyst system of the present invention can tolerate recycle stream containing 10 to 14 wt % of p,p-BPA, 2 to 4 wt % of o,p-BPA, and 4 to 8 wt % of other BPA impurities, without reducing the lifetime of the catalyst system. In another aspect, the DMT attached promoter catalyst system can tolerate other impurities detrimental to conventional catalyst systems. In yet another aspect, the DMT attached promoter catalyst system can provide performance equivalent to or greater than that of conventional bulk promoter systems. In another aspect, the DMT promoter catalyst system can prevent the need for a separate purification step for process recycle streams.

In one aspect, when using a recycled phenol stream, the DMT catalyst system can provide levels of p,p-BPA that are within about 10%, within about 8%, within about 6%, within about 4%, or within about 2% of values obtained using a fresh phenol stream. In a specific aspect, when using a recycled phenol stream, the DMT catalyst system can provide levels of p,p-BPA that are within about 5% of values obtained using a fresh phenol stream.

Thus, in various aspects, the DMT catalyst system can tolerate recycle stream impurities with no significant degradation in catalyst performance.

### Selectivity

As briefly noted above, the condensation of phenol and acetone to form BPA can yield multiple isomers of BPA, together with other reaction products. For most applications, the p,p-BPA isomer is preferred over the o,p-BPA isomer. In a conventional BPA manufacturing process using a bulk promoter system, isomerization of the BPA reaction product occurs until an equilibrium is reached. The amount of each isomer present at equilibrium depends on the temperature of the reaction medium, as detailed in Table 1, below.

TABLE 1
Equilibrium BPA isomer ratio

| Temperature (° C.) | Equilibrium pp/op ratio |
|---|---|
| 50 | 14.6/1 |
| 60 | 11.6/1 |
| 70 | 10.1.1 |
| 80 | 8.9/1 |
| 90 | 8.1/1 |
| 100 | 6.8/1 |

For conventional bulk promoter systems, higher temperatures can accelerate the reaction rate, but can also accelerate isomerization and the proportion of undesirable o,p-BPA present. Thus, separate isomerization reactors are typically needed to convert produced o,p-BPA to the preferred p,p-BPA isomer. In bulk promoter systems, the isomerization reactor can typically utilize a highly cross-linked (greater than about 8%) ion exchange resin to convert o,p-BPA to p,p-BPA.

Bulk promoter systems typically provide a p,p/o,p-BPA ratio of 10 to 15. In one aspect, the DMT catalyst system can exhibit a higher p,p-BPA to o,p-BPA ratio than a conventional bulk promoter system. In various aspects, the p,p/o,p ratio for the DMT catalyst system can be at least about twice that for conventional bulk promoter systems. In various aspects, a DMT catalyst system can exhibit a p,p/o,p BPA ratio of at least about 20, at least about 30, at least about 40, at least about 50, at least about 60, or more. In another aspect, a DMT catalyst system can exhibit a p,p/o,p-BPA ratio of at least about 25, for example, about 25, 26, 27, 28, 29, 30, 31, 32, 33, 34, 35, 40, or more. In yet another aspect, a DMT catalyst system (22% attachment) can exhibit a p,p/o,p-BPA ratio of from about 25 to about 35.

In another aspect, the improved selectivity of the DMT catalyst system can eliminate the need for a separate isomerization process.

In various aspects, the inventive DMT catalyst system can provide simplified methods for catalyzing condensation reactions. In one aspect, the present invention provides a process for catalyzing a condensation reaction that utilizes a modified ion exchange resin catalyst having an attached dimethyl thiazolidine promoter. In another aspect, the present invention provides a process for catalyzing a condensation reaction that does not utilize a bulk promoter system.

In one aspect, the inventive DMT catalyst system can allow a simplified BPA manufacturing process, wherein one or more of the following are not needed: phenol pretreatment/purification step, acetone pretreatment/purification step, BPA recycle stream purification step, separate isomerization reaction, or a combination thereof. In other aspects, a manufacturing process comprising the inventive DMT catalyst can provide an efficient, selective, longer lifetime catalyst system than conventional attached promoter catalyst systems.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

1. Hydroxyacetone Tolerance

In a first example, a single column reactor was utilized to determine the inventive catalyst system's tolerance for hydroxyacetone (HA) impurities. Parallel reactions were performed: one with 20 ppm HA present in the phenol reactant, the other without HA in the phenol reactant. Reactions were carried out at 75° C., for 100 hours, using 7.5 wt. % acetone, and at WHSV of 20. The ion exchange resin utilized was Lanxess K1221 SH, modified to a level of 20% with the inventive DMT promoter.

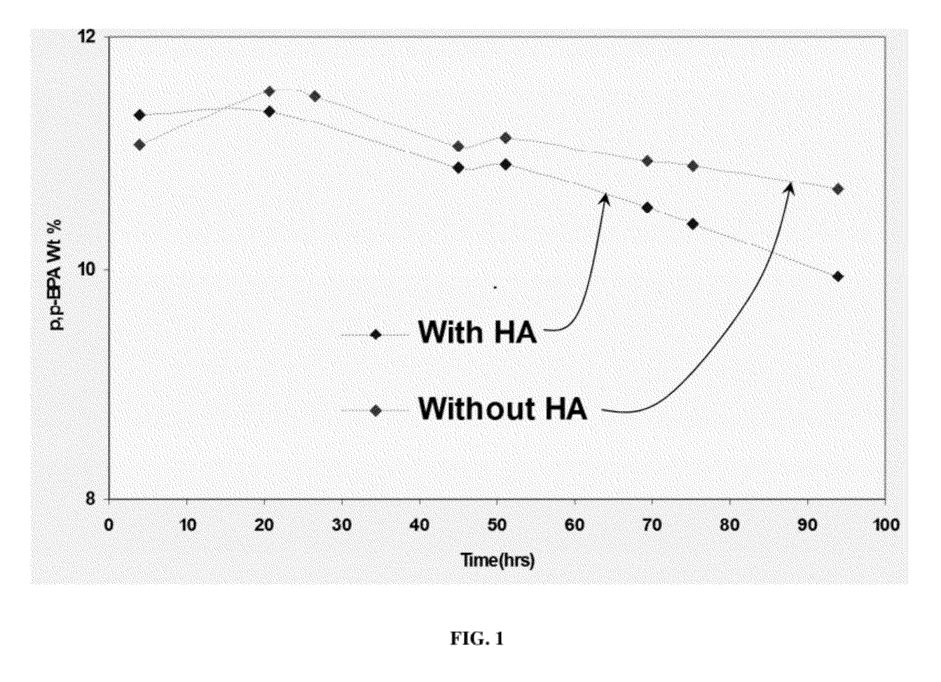

The amount of p,p-BPA produced was then monitored over time. As illustrated in FIG. 1, the reaction occurring in the presence of HA exhibited nearly identical performance to the reaction without HA. After 94 hours, the amount of acetone converted to p,p-BPA was 41% in the reaction without HA, and 38% in the reaction with HA.

The reduction in acid strength (meq/g) of the catalyst system after the 100 hour test was 11.04% for the reaction without HA vs. 15.41% for the reaction with HA present. Thus, only a 4.37% difference in catalyst acid strength was observed between the HA and HA free reactions after 100 hours of operation.

2. Methanol Spiking

In a second example, BPA synthesis experiments were performed, wherein the acetone reactant was spiked with methanol. In a first spiking experiment, a single column reactor was operated (WHSV=2, 65° C.) in continuous fashion with an acetone concentration of about 5%. The amount of p,p-BPA formed was monitored over time, as the column feed was periodically spiked with various levels of methanol.

FIG. 2 illustrates the amount of p,p-BPA produced as the column was spiked with methanol (550 ppm, 3157 ppm, and 110 ppm). The observed deactivation profile was identical to that expected when no methanol is present. Thus, the presence of methanol has no detectable effect on the performance of the catalyst system and the formation of p,p-BPA.

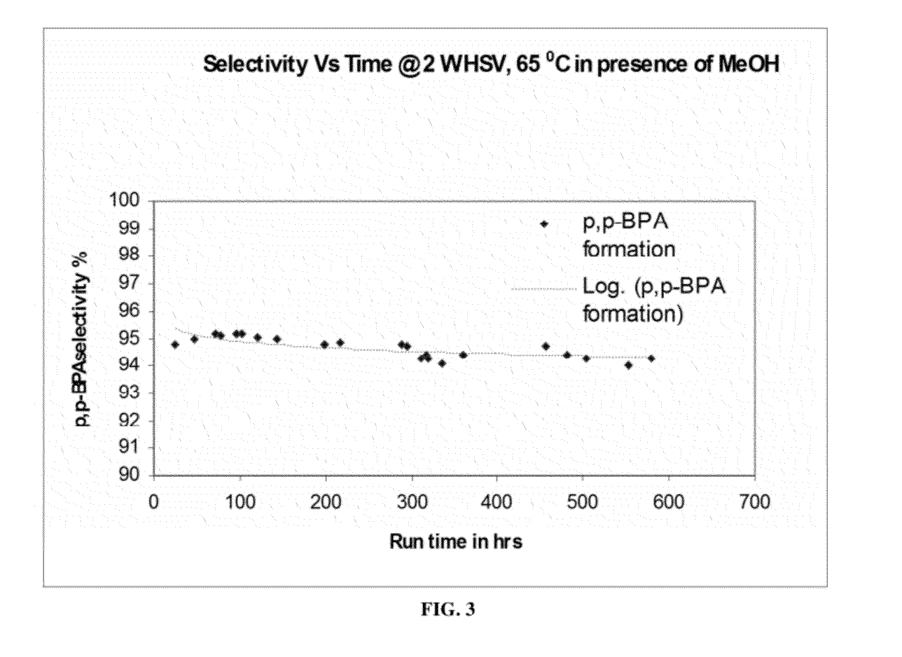

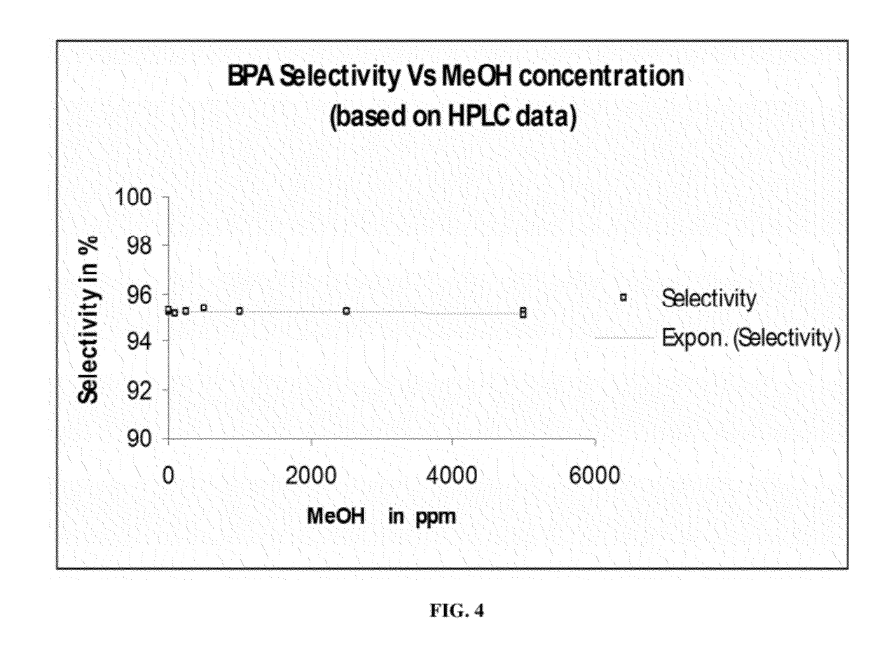

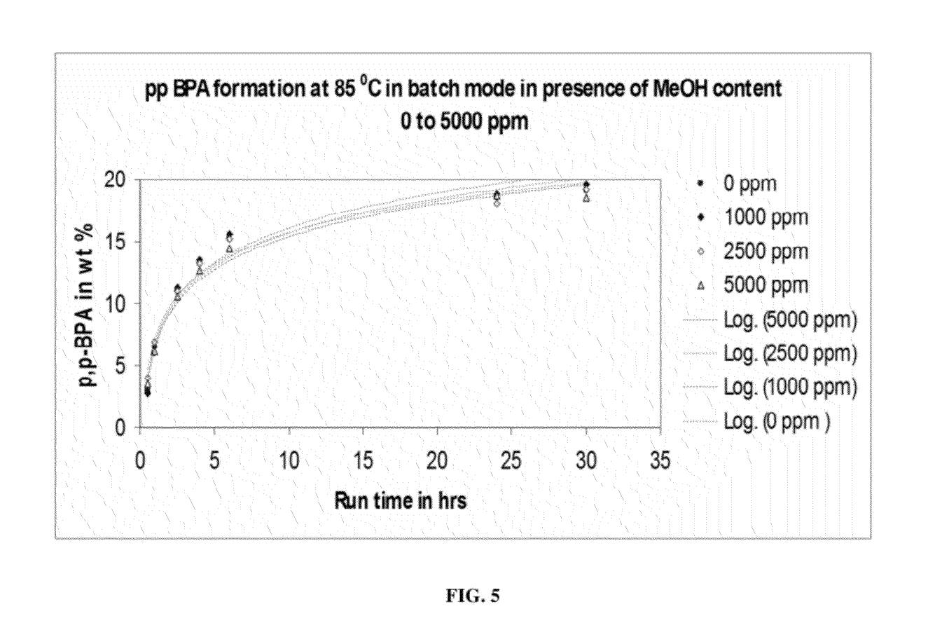

Similarly, FIG. 3 illustrates the selectivity of the inventive catalyst system in the same methanol spiking experiment illustrated in FIG. 2The presence of methanol in the reaction did not have an effect on the high selectivity of the DMT catalyst towards p,p-BPA. In a separate batch reaction using 5.59% acetone (4 hours at 65° C.), the amount of methanol present in the system was varied between 0 and 5,000 ppm. The selectivity was then monitored as the concentration of methanol in the system varied. As illustrated in FIG. 4, the inventive DMT catalyst system exhibited virtually no change in selectivity over the varying concentration range of methanol.

In yet another set of batch reactions (5.59 wt. % acetone, 4 hours at 65° C.), one reaction was conducted with no methanol present, whereas the second reaction had 1.27 ml of methanol added to the reactants. The concentration of specific reaction products was then determined. The amount of o,p-BPA produced with no methanol present was 0.279%, compared to 0.298% when methanol was added. Similarly, the amount of p,p-BPA produced with no methanol present was 9.935%, compared to 10.667% when methanol was added. Thus, the addition of methanol with the DMT catalyst system had no adverse effect on the production of p,p-BPA at 65° C.

In another batch reaction conducted at 85° C. (5.59 wt. % acetone, 30 hours), a series of individual reactions were performed at varying methanol concentrations ranging from 0 to 8.94 wt. %. The amount of p,p-BPA produced over time was measured for each reaction, and is illustrated in FIG. 5. Thus, the inventive DMT catalyst system can remain unaffected by up to at least about 8.9% methanol.

3. Isomerization of O,P-BPA in Attached Promoter System

In a third example, a single column reactor was operated (WHSV 1 and 2) at 65° C. and 75° C. with a reactant feed of 4.5 wt. % acetone and phenol with 2% o,p-BPA. The catalyst system comprised a 2% cross-linked A121 ion exchange resin with 22% attached dimethyl thiazolidine (DMT).

As detailed in Table 2, below, the DMT catalyst provides effective isomerization and selectivity for the production of p,p-BPA. The DMT catalyst provided a high ratio of p,p-BPA/o,p-BPA and a high degree of selectivity. It should also be noted that isomerization to p,p-BPA increases with increasing o,p-BPA content in the reactor, indicating the usefulness of the inventive catalyst system for acting as a stand-alone catalyst, without the need for a separate isomerization reactor.

TABLE 2
Isomerization Experiment Data

| | Temp, ° C. | | | | | |
|---|---|---|---|---|---|---|
| | 65 | 75 | 65 | 75 | 65 | 75 |
| WHSV | 1.00 | 1.00 | 2.00 | 2.00 | 2.00 | 2.00 |
| % o,p-BPA | 1.00 | 1.00 | 1.00 | 1.00 | 2.00 | 2.00 |
| p,p/o,p- | 28.67 | 23.78 | 32.16 | 27.87 | 64.23 | 42.69 |
| BPA (diff) | | | | | | |
| Selectivity | 95.20 | 94.40 | 95.57 | 94.98 | 96.67 | 95.78 |

What is claimed is:

1. A catalyst system comprising a cross-linked, sulfonated ion exchange resin catalyst and a dimethyl thiazolidine promoter.

2. The catalyst system of claim 1, wherein the cross-linked, sulfonated ion exchange resin comprises a plurality of sulfonic acid groups and has a degree of cross-linking of from about 1% to about 4%.

3. The catalyst system of claim 1, wherein the cross-linked, sulfonated ion exchange resin has a degree of cross-linking of from about 1.5% to about 2.5%.

4. The catalyst system of claim 1, wherein the cross-linked, sulfonated ion exchange resin has a degree of cross-linking of about 2%.

5. The catalyst system of claim 1, wherein the dimethyl thiazolidine promoter is at least partially bound to the cross-linked, sulfonated ion exchange resin.

6. The catalyst system of claim 1, wherein at least a portion of the dimethly thiazolidine promoter is covalently bound to the cross-linked, sulfonated ion exchange resin.

7. The catalyst system of claim 1, wherein at least a portion of the dimethyl thiazolidine promoter is ionically bound to the cross-linked, sulfonated ion exchange resin.

8. The catalyst system of claim 2, wherein the dimethyl thiazolidine promoter is bound to from about 18% to about 25% of the sulfonic acid groups of the cross-linked, sulfonated ion exchange resin.

9. The catalyst system of claim 2, wherein the dimethyl thiazolidine promoter is bound to from about 20% to about 24% of the sulfonic acid groups of the cross-linked, sulfonated ion exchange resin.

10. The catalyst system of claim 2, wherein the dimethyl thiazolidine promoter is bound to about 22% of the sulfonic acid groups of the cross-linked, sulfonated ion exchange resin.

11. The catalyst system of claim 1, wherein the cross-linked, sulfonated ion exchange resin comprises a polystyrene based ion exchange resin.

12. The catalyst system of claim 1, wherein the cross-linked, sulfonated ion exchange resin comprises a polystyrene based ion exchange resin having a degree of cross-linking of from about 1% to about 4%, and wherein at least a portion of the dimethyl thiazolidine promoter is ionically bound to the ion exchange resin.

13. The catalyst system of claim 1, wherein the cross-linked, sulfonated ion exchange resin comprises a polysiloxane based ion exchange resin.

14. The catalyst system of claim 1, wherein the cross-linked, sulfonated ion exchange resin comprises a polysiloxane based ion exchange resin having a degree of cross-linking of from about 1% to about 4%, and wherein at least a portion of the dimethyl thiazolidine promoter is ionically bound to the ion exchange resin.

15. An attached promoter catalyst system comprising an ion exchange resin and a dimethyl thiazolidine promoter, wherein the catalyst system is more resistant to hydroxyacetone than a conventional bulk promoter system.

16. The attached promoter catalyst system of claim 15, wherein the catalyst system can maintain at least about 60% of its initial performance after 200 hours of operation in the presence of about 10 ppm hydroxyacetone.

17. The attached promoter catalyst system of claim 15, wherein the catalyst system can maintain at least about 75% of its initial performance after 200 hours of operation in the presence of about 10 ppm hydroxyacetone.

18. The attached promoter catalyst system of claim 15, wherein the catalyst system is substantially unaffected, during operation, by exposure to up to about 1,000 ppm of alcohol.

19. The attached promoter catalyst system of claim 15, wherein the catalyst system is substantially unaffected, during operation, by exposure to up to about 3,000 ppm of alcohol.

20. The attached promoter catalyst system of claim 15, wherein the catalyst system is substantially unaffected by recycled bisphenol-A impurities.

21. The attached promoter catalyst system of claim 15, wherein the catalyst system, in a condensation reaction, can selectively catalyze the production of p,p-bisphenol-A in a ratio of at least about 25:1 (p,p-bisphenol-A:o,p-bisphenol-A).

22. The attached promoter catalyst system of claim 15, wherein the catalyst system, in a condensation reaction, can selectively catalyze the production of p,p-bisphenol-A in a ratio of at least about 30:1 (p,p-bisphenol-A:o,p-bisphenol-A).

23. A method for catalyzing a condensation reaction, the method comprising contacting two or more reactants with a modified ion exchange resin catalyst in the absence of a bulk promoter.

24. The method of claim 23, wherein the method does not utilize a 3-mercaptopropionic acid bulk promoter.

25. The method of claim 23, wherein the modified ion exchange resin catalyst comprises a cross-linked, sulfonated ion exchange resin.

26. The method of claim 25, wherein the cross-linked, sulfonated ion exchange resin comprises a plurality of sulfonic acid groups and has a degree of cross-linking of from about 1% to about 4%.

27. The method of claim 25, wherein the cross-linked, sulfonated ion exchange resin has a degree of cross-linking of from about 1.5% to about 2.5%.

28. The method of claim 25, wherein the cross-linked, sulfonated ion exchange resin has a degree of cross-linking of about 2%.

29. The method of claim 25, wherein the modified ion exchange resin catalyst comprises an attached dimethyl thiazolidine promoter.

30. The method of claim 29, wherein the dimethyl thiazolidine promoter is at least partially bound to the cross-linked, sulfonated ion exchange resin.

31. The method of claim 29, wherein at least a portion of the dimethly thiazolidine promoter is covalently bound to the cross-linked, sulfonated ion exchange resin.

32. The method of claim 29, wherein at least a portion of the dimethyl thiazolidine promoter is ionically bound to the cross-linked, sulfonated ion exchange resin.

33. The method of claim 29, wherein the dimethyl thiazolidine promoter is bound to from about 18% to about 25% of the sulfonic acid groups of the cross-linked, sulfonated ion exchange resin.

34. The method claim 29, wherein the dimethyl thiazolidine promoter is bound to from about 20% to about 24% of the sulfonic acid groups of the cross-linked, sulfonated ion exchange resin.

35. The method of claim 29, wherein the dimethyl thiazolidine promoter is bound to about 22% of the sulfonic acid groups of the cross-linked, sulfonated ion exchange resin.

36. The method of claim 29, wherein the cross-linked, sulfonated ion exchange resin comprises a polystyrene based ion exchange resin.

37. The method of claim 29, wherein the cross-linked, sulfonated ion exchange resin comprises a polystyrene based ion exchange resin having a degree of cross-linking of from about 1% to about 4%, and wherein at least a portion of the dimethyl thiazolidine promoter is ionically bound to the ion exchange resin.

38. The method of claim 29, wherein the cross-linked, sulfonated ion exchange resin comprises a polysiloxane based ion exchange resin.

39. The method of claim 29, wherein the cross-linked, sulfonated ion exchange resin comprises a polysiloxane based ion exchange resin having a degree of cross-linking of from about 1% to about 4%, and wherein at least a portion of the dimethyl thiazolidine promoter is ionically bound to the ion exchange resin.

40. The method of claim 23, wherein the two or more reactants comprise a phenol and at least one of a ketone, an aldehyde, or a combination thereof.

41. A method for the production of bisphenol-A, the method comprising contact a phenol and at least one of a ketone, an aldehyde, or a combination thereof in the presence of an attached ion exchange resin catalyst comprising a dimethyl thiazolidine promoter, wherein the method does not comprise a pretreatment and/or purification step for the phenol, ketone, and/or aldehyde.
